SEQUENCE LISTING (12 sequences)

SEQ ID NO: 1 - DNA, 2208 bp, Foot-and-mouth disease virus
atgggggcagggcagtcatcaccagccacaggaagtcagaatcagagcggcaacaccgggtccatcatcaacaactactatatgcagcagtatcagaactctatggacacccagctgggcgataacgccatctctggcggctctaatgagggctctacagatacaacctctacacacaccacaaatacccagaataacgactggttttcccggctggccagctctgccttctctggcctgtttggcgccctgctggccgacaagaagaccgaggagacaaccctgctggaggatagaatcctgacaaccaggaacggccacaccaccagcacaacccagtcctctgtgggcgtgacctacggctactctacaaccgaggaccacgtggccggcccaaatacctccggcctggagacacgggtggtgcaggccgagagattctacaagaagttcctgtttgactggaccaccgataagccctttggccacctgcacaagctggagctgccaacagatcactacggcgtgttcggccacctggtggactcctatgcctacatgagaaacggctgggacgtggaggtgtccgccgtgggcaaccagttcaacggcggctgcctgctggtggccatggtgccagagtggaaggagttcgagacaagggagaagtaccagctgaccctgtttccccaccagtttatctcccctagaaccaatatgaccgcccacatcacagtgccatacctgggcgtgaaccggtacgaccagtaccggaagcacaagccctggaccctggtggtaatggtggtgtcccccctgacagccaatgccacatccgccgcccagatcaaggtgtacgccaatatcgcccctacatacgtgcacgtggccggcgagctgccttccaaggagggcatcttccctgtggcctgtgccgatggctatggcggcctggtgacaaccgatcccaagaccgccgatcctgcctatggcaaggtgtacaacccccctcggaccaactatcctggccggtttaccaacctgctggatgtggccgaggcctgtcccacctttctgtgcttcgatgatggcaagccctacgtgacaacccagaccggcgagtcccgcctgctggccaagtttgacctgtccctggccgccaagcacatgtccaacacctatctggccggcatcgcccagtactatacccagtacagcggcaccatcaacctgcacttcatgttcacaggctctacagactccaaggccagatatatggtggcctatatccccccaggcgtggagacacccccagataccccagagagggccgcccactgtatccacgccgagtgggataccggcctgaacagcaagttcacattttccatcccttacgtgtctgccgccgactatgcctataccgccagcgatgaggccgagaccacaaacgtgcagggctgggtgtgcgtgtaccagatcacccacggcaaggccgacaatgatacactggtggtgtccgtgagcgccggcaaggatttcgagctgagactgcctatcgaccctcggccccagacaaccgccacaggcgagagcgccgatcctgtgaccacaacagtggagaattatggcggcgagacccaggtgcagagacgccaccacaccgatgtgggcttcatcatggacagatttgtgaagatcaactccccaaagagcacccacgtgatcgatctgatgcagacccaccagcacggcctggtgggcgccctgctgagagccgccacatattacttctccgatctggagatcgtggtgcggcacgacggcaatctgacatgggtgcccaatggcgccccagtgagcgccctgtccaacacatccaaccctaccgcctacaacaaggcccctttcacaaggctggccctgccttacaccgccccacaccgcgtgctggccaccgtgtacaacggcacctccaagtacaccgtgtccggcctgtccagaagaggcgacctgggctccctggccgccagggtggccaaggccctgccagcctcttttaactatggcgccatcaaggccgtgaatgtgcacgagctgctggtgcggatgaagagggccgagctgtactgtcccagaccactgctggccctggaggtaatctcccaggatcggcacaagcagaagatcatcgccccagagaaacag

SEQ ID NO: 2 - DNA, 693 bp, Foot-and-mouth disease virus
atgagtggtgccccaccgaccgacctgcagaagatggtcatgggcaacacaaagcctgttgagcttattctcgacgggaagacagtagccatctgctgcgctactggagtgttcggcactgcctacctcgtgcctcgtcatcttttcgctgagaagtacgacaagatcatgctggacggcagagccctgacagacagtgactacagagtgtttgagtttgagattaaagtaaaaggacaggacatgctctcagacgctgcgctcatggtgcttcaccgtgggaaccgcgtgagagacatcacgaaacactttcgtgacacagctagaatgaagaaaggcacccccgttgttggcgttatcaacaacgctgacgtcgggagactgatcttctctggtgaagcccttacctacaaggacattgtagtgtgcatggacggagacaccatgccagggctttttgcctacaaagccgcaactaaggctggctattgcggaggggccgttctcgcaaaggacggagccgacactttcatcgttggcacccactctgctggaggcaatggagttggttactgctcatgcgtttccaggtccatgctccaaaagatgaaggcacacgtcgacccggagccacaccacgaagggttgattgttgacaccagagatgtggaagaacgagtccacgtgatgtaa

SEQ ID NO: 3 - DNA, 56 bp, Artificial Sequence
  Apaf
taggcgcaaaggcttggctcatggttgacagctcagagagagaaagatctgaggga

SEQ ID NO: 4 - DNA, 33 bp, Artificial Sequence
  CR1
aacaaacagacaatctggtctgtttgtattata

SEQ ID NO: 5 - DNA, 184 bp, Artificial Sequence
  CR5
aacagacaatctggtctgtttgtattataacaaacagacaatctggtctgtttgtattataacaaacagacaatctggtctgtttgtattataacaaacagacaatctggtctgtttgtattataacaaacagacaatctggtctgtttgtattataacaaacagacaatctggtctgtttgta

SEQ ID NO: 6 - DNA, 42 bp, Artificial Sequence
  OG521
aaacaattgggccggccaccatgagtggtgccccaccgaccg

SEQ ID NO: 7 - DNA, 42 bp, Artificial Sequence
  OG522
aaacaattgggccggccaccacgagtggtgccccaccgaccg

SEQ ID NO: 8 - DNA, 52 bp, Artificial Sequence
  OG523
aaaagatctacgcgttcattacatcacgtggactcgttcttccacatctctg

SEQ ID NO: 9 - DNA, 170 bp, Artificial Sequence
  OG524
aaagcggccgctccactattcgaggccgttcgttaatacttgttgcgttcctagccgctattaggcgcaaaggcttggctcatggttgacagctcagagagagaaagatctgagggaggccggccgccaccatgagtggtgccccaccgaccgacctgcagaagatggtc

SEQ ID NO: 10 - DNA, 46 bp, Artificial Sequence
  OG525
aaaggatcctcattacatcacgtggactcgttcttccacatctctg

SEQ ID NO: 11 - DNA, 170 bp, Artificial Sequence
  OG526
aaagcggccgctccactattcgaggccgttcgttaatacttgttgcgttcctagccgctattaggcgcaaaggcttggctcatggttgacagctcagagagagaaagatctgagggaggccggccgccaccacgagtggtgccccaccgaccgacctgcagaagatggtc

SEQ ID NO: 12 - DNA, 2208 bp, Foot-and-mouth disease virus
atgggggctggacaatccagcccagctactggctcgcagaaccaatctggtaacacaggtagcataatcaacaactactacatgcaacagtaccaaaactccatggacacacagcttggtgacaatgccatcagtggaggctctaacgagggctccacggacacaacttcaactcacacaaccaacacccaaaacaatgactggttttcaagactcgccagttcggccttctccggtttgtttggggccttgcttgccgacaagaagacggaggagacgacactccttgaggaccgcattctcaccactcgtaatgggcacaccacctccacgacccagtccagcgtaggcgttacatacgggtactccacaacagaggaccacgttgctggacccaacacatcaggtttggagacacgagtggtacaggcagagagattctacaaaaagtttttgtttgattggacaacggacaagccttttggacacctgcacaaactggagttgcccaccgaccactacggtgttttcggacacttggtggactcatacgcctacatgaggaacggttgggacgttgaggtgtctgctgttggcaaccagttcaacggcggatgcctcctagtggccatggtacccgaatggaaagagtttgaaacgcgggagaagtaccagctcacgcttttcccgcaccagttcattagccccagaaccaacatgactgcccacatcacggttccttaccttggtgtgaatagatatgatcagtacagaaaacacaaaccctggacactggttgtcatggtcgtgtccccgctcacggccaacgccacgagcgcggcacagatcaaggtctatgccaacatcgctccgacctacgttcatgtggccggcgagctcccctcgaaagaggggatcttccctgtcgcgtgcgcggacggttacggaggactggtgacaacggacccgaaaacagctgaccccgcctacggcaaggtgtacaatccgccccggactaactaccccgggcgtttcaccaacttgttggacgtggctgaggcatgtcccacctttctgtgttttgacgacgggaaaccgtacgttaccacacagacaggtgagtctcgtcttctggccaagttcgacctttcccttgccgcgaagcacatgtctaacacatatttggcaggaattgcccagtactacacacagtactcgggcaccatcaatttgcatttcatgttcacaggttcaactgattcaaaagcccgctacatggtggcttacatcccgcctggggtggaaacaccaccggatacacctgagagggcagcccactgcatccatgctgagtgggacacagggttgaattccaaattcacattctcaatcccgtacgtgtctgccgcggattacgcctacacggcgtctgatgaggcagagacaacaaacgtacagggatgggtctgcgtttaccagatcacacacgggaaggctgacaacgacactctggtcgtatcggttagcgccggcaaggacttcgagttgcgcctccccattgacccccgaccgcagaccaccgctactggggaatcagcagaccctgtcaccaccactgtagagaactacggcggtgagacacaagttcagagacgccaccacaccgacgttggcttcatcatggacagatttgtgaaaataaacagcccaaaatccacccatgtcattgacctcatgcaaacccaccaacacggtctagtgggtgcgctgctgcgtgcggcgacctactacttctcagatctggaaattgttgtgcggcacgacggtaacctaacttgggtgcccaatggtgctcccgtgtcagccttgtccaacaccagcaaccccaccgcctacaacaaggcaccgttcacgagacttgccctcccctacaccgcgccacaccgcgtgttggcgactgtgtacaacgggacgagcaagtacactgtgagtgggttaagcagacgaggcgacttgggttccctcgcggcacgagtcgcgaaggcacttcctgcttctttcaactacggtgcaatcaaggccgtcaacgtgcacgagcttctcgtgcgcatgaaacgggccgaactctactgccctagaccactgttggcactagaggtgatctcacaagacagacacaaacagaagatcattgcacccgaaaaacag

(19) United States
(12) Patent Application Publication
(10) Pub. No.: US 2024/0010686 A1
(43) Pub. Date: Jan. 11, 2024

(54) DNA CONSTRUCT FOR STABLY PRODUCING EMPTY CAPSIDS OF THE FOOT-AND-MOUTH DISEASE VIRUS IN MAMMALIAN CELLS; PROCESSES, USES, AND COMPOSITIONS THEREOF

(71) Applicants: Instituto Nacional de Tecnologia Agropecuaria, Ciudad Automoma de Buenos Aires (AR); National Research Council of Canada, Ottawa (CA)

(72) Inventors: Ana Clara Mignaqui, Rio Negro (AR); Andres Wigodorovitz, Buenos Aires (AR); Yves Durocher, Montreal (CA)

(73) Assignees: Instituto Nacional de Tecnologia Agropecuaria, Ciudad Automona de Buenos Aires (AR); National Research Council of Canada, Ottawa, ON (CA)

(21) Appl. No.: 18/252,212

(22) PCT Filed: Nov. 9, 2021
(86) PCT No.: PCT/CA2021/051592

§ 371 Date: May 9, 2023

(30) Foreign Application Priority Data
Nov. 9, 2020 (AR) P20200103102

(51) Int. Cl. C07K 14/005; C12N 9/64; C12N 15/63; C12N 7/04

(52) U.S. Cl. CPC C07K 14/005; C12N 9/641; C12N 15/63; C12N 7/045; C12Y 304/22028

(57) ABSTRACT

Methods and compositions for increasing the production of large amounts of empty capsids of the foot-and-mouth disease virus (FMDV) in a stable manner in mammalian cells by regulating the expression of FMDV 3C protease. The instant methods and compositions are based on the fact that a decreased expression of 3C protease results in a reduced cell toxicity and an increased synthesis of viral capsid proteins, as well as production of recombinant empty capsids. The invention provides recombinant plasmids that direct the expression of P1, 3C, and the use of the plasmids for producing new stable cell lines capable of generating high titers of FMDV empty capsids. The invention provides methods for regulating the expression of the FMDV 3C protease gene at a transcriptional and translational level in order to achieve the required process level of 3C protease for the selection process, as well as the production process.

### CROSS REFERENCE TO RELATED APPLICATION

This application is a national stage entry of PCT/CA2021/051592 filed Nov. 9, 2021, under the International Convention claiming priority over Argentina Patent Application No. P20200103102 filed Nov. 9, 2020.

### FIELD OF THE INVENTION

The present invention refers to a DNA construct for stably producing empty capsids of the foot-and-mouth disease virus in mammalian cells comprising: a) a nucleotide sequence encoding a P1 polyprotein, b) a nucleotide sequence encoding a 3C protease, wherein the nucleotide sequence encoding the 3C protease comprises a modified translation start codon; processes, and uses thereof.

### BACKGROUND OF THE INVENTION

The foot-and-mouth disease (FMD) is a highly contagious disease of cloven-hoofed animals such as cattle, pigs, sheep and goats, that even affects deer and wild boars. The disease is endemic in many parts of the developing world and still represents a serious threat to livestock industries. It is of economic importance as the presence of the disease in developing countries leads to severe restrictions on international trade, and an outbreak in countries free of foot-and-mouth disease can cause billion-dollar losses. (Thompson D, et al., (2002) Rev Sci Tech 21: 675-687). Prevention and eradication of the disease in a country requires a continuous effort at a high cost. Vaccination is still an important strategy in developing countries to control foot-and-mouth disease and has become relevant as an alternative to control outbreaks in places that are free of the disease (Parida S (2009) Expert Rev Vaccines., 8(3): 347-65).

The foot-and-mouth disease virus (FMDV), a member of the Picornaviridae family, Aphthovirus genus, is a non-enveloped, singled stranded positive sense RNA virus and the causative agent of the disease (Grubman M & Baxt B (2004) Clin Microbiol Rev., 17(2): 465-493). The FMDV has an icosahedric capsid formed by assembly of the structural proteins VP1, VP2, VP3, and VP4. The P1 capsid precursor is processed by a 3C protease to produce structural proteins VP0, VP3, and VP1. A copy of each of these proteins spontaneously assemble to form a protomer (5S), then five protomers form a pentamer (12S) and finally twelve pentamers assemble forming an empty capsid (75S). The cleavage of VP0 into VP2 and VP4 occurs after encapsidation of the genomic RNA in the empty capsids, thus resulting in a complete virion (146S).

Seven FMDV serotypes (A, O, C, SAT1, SAT2, SAT3, and ASIA1) have been described, with multiple subtypes within each serotype. A viral infection or vaccination with a serotype does not confer protection against other serotypes. Accordingly, an update of the antigenic composition of the vaccine is needed, considering the epidemiology of the site and the appearance of new field strains.

The currently employed vaccine for controlling FMD consists in a chemically inactivated virus generally formulated with an oil adjuvant. In order to produce this vaccine, mammalian cells (BHK-21) grown in a suspension infected with virus are used, and then binary ethyleneimine is used for the inactivation process (Doel T R (2003) Virus Res., 91(1): 81-99). Although the inactivated vaccine is effective in the control and eradication of the foot-and-mouth disease there is a series of drawbacks associated with its use and production. On one hand, the need of high biosafety production facilities and the risk of an incomplete inactivation of the virus. On the other hand, there is a problem with distinguishing vaccinated from infected animals, unless purification methods are included in the productive system, and the fact that some serotypes and subtypes are difficult to grow in cell culture. To address these problems, there is a need for developing new vaccines to generate immunity, as efficient as the inactivated virus, but safer (Diaz-San Segundo F, et al., (2017) Vet Microbiol 206: 102-112).

Recombinant FMDV empty capsids represent a promising alternative for developing new vaccines as they mimic the viral structure, but lack the infectious nucleic acid (Belsham G J and Bøtner A (2015) Virus Adapt Treat 7: 11-23). Immunogenicity and safety of the FMDV empty capsids have already been widely demonstrated, however achieving a scalable and economical production is a critical aspect which must also be further explored (Mignaqui A C, et al., (2019) Crit Rev Biotechnol 39: 306-320). In this regard, there is a great number of ongoing approaches for expressing recombinant empty capsids based on complex technologies that involve many steps and difficulties for scaling up, all of which limit the amount of doses to be produced.

These technologies may be applied in emergency vaccines for controlling outbreaks in disease-free zones because the number of doses required in these cases is limited, but they are impractical or very hard to implement in order to meet the demand of doses needed to be produced for controlling the disease in endemic zones.

The stable production of empty capsids of the foot-and-mouth disease virus in mammalian cells would be the ideal strategy as it requires the same infrastructure that currently exists in virus production facilities which are used for the inactivated vaccine. The traditional protein production in mammalian cells involves generating stable lines producing the recombinant protein of interest (Durocher Y, Butler M (2009) Current Opinion in Biotechnology, 20: 700-707; Wurm F M (2004) Nature Biotechnology; 22: 1393-1398). To this end, it is necessary to clone the construct encoding the chosen protein into a suitable vector, introduce it in the cells and then integrating it into the cell-line genome during the selection process. Once a recombinant cell line is obtained, it represents an unlimited source of recombinant proteins only produced by culture of the cells, thus ensuring repeatability among batches. Despite the advantages of mammalian cells as an expression system, the recombinant stable production of empty FMDV capsids in mammalian cells has remained little explored. This is mainly due to difficulties related to isolating stable high expression level cell lines because of the toxicity of the 3C protease, that not only cleaves the P1 capsid precursor but also cell proteins (Falk M M, et al (1990) J Virol 64: 748-756; Belsham G J, et al (2000) J Virol 74: 272-280). In fact, the attempts to generate a recombinant cell line stably expressing the proteins required for the assembly of the empty capsid have repeatedly failed, as the 3C protease impairs the isolation of high expression cells or clones (Mignaqui A C, et al., (2013) Advances in Bioscience and Biotechnology 4: 1024-1029).

The deleterious effect of 3C protease has not only affected the development of stable lines of mammalian cells expressing the empty FMDV capsids but also the yield of empty capsids obtained using other expression systems or strategies. Although strategies for improving the expression of polyproteins of the foot-and-mouth disease virus or other virus by controlling or modifying the 3C protease are known, for example, from documents WO2018/048652, WO2019226190, EP2491118, EP1007637, none of them have made it possible to stably produce empty FMDV capsids in mammalian cells.

The recombinant FMDV empty capsids have been produced in heterologous expression systems, such as bacteria, baculovirus, and transgenic plants. However, due the toxicity of the 3C protease, so far it has not been possible to use mammalian cells in the commonly known way of producing biotherapeuticals, that involves developing stable cell lines, (Lalonde M E and Durocher Y, (2017) Journal of Biotechnology; 251: 120-140; Wurm F, (2004) Nature Biotechnology, 22(11): 1393-1398). The production of VLPs in mammalian cells has only been reported using transient expression with methodologies that are not viable for a large scale approach of a vaccine for the foot-and-mouth disease (Mignaqui A C, et al., (2013) Plos One, 8(8), p.e 72800; Gullberg M, et al., (2013) Journal of General Virology; 94: 1769-1779). In addition, the yields achieved so far with mammalian cells are the lowest among recombinant expression systems.

### SUMMARY OF THE INVENTION

The invention provides a DNA construct for stably producing empty capsids of the foot-and-mouth disease virus in mammalian cells that comprises: a) a nucleotide sequence encoding a P1 polyprotein, b) a nucleotide sequence encoding a 3C protease, wherein the nucleotide sequence encoding the 3C protease comprises a modified translation start codon. In a preferred embodiment, the nucleotide sequence encoding the P1 polyprotein has a sequence identity of from 70% to 99% to SEQ ID NO:12 or from 90 to 99% of SEQ ID NO:1; and the nucleotide sequence encoding 3C protease has a sequence identity of from 90% to 99% to SEQ ID NO:2.

In one embodiment, the nucleotide sequence encoding a P1 polyprotein has a sequence identity of at least 70%, at least 75%, at least 80%, at least 85%, at least 90%, at least 95%, at least 96%, at least 97%, at least98% or at least 99% to full-length SEQ ID NO:12. In an embodiment, the nucleotide sequence encoding a 3C protease has a sequence identity of at least 90%, at least 95%, at least 96%, at least 97%, at least 98%, or at least 99% to full-length SEQ ID NO:2.

The inducible promoter that regulates 3C may be the coumate-inducible promoter (CR1) or any other inducible promoter. The inducible promoter that regulates P1 may be a coumate-inducible promoter (CR5) or any other inducible or non-inducible promoter. The construct comprises a modification of the sequence, wherein the translation start codon ATG has been changed to ACG. Inducible promoters may be CR5—a coumate-inducible promoter, CR1—a coumate-inducible promoter and TRE—inducible by tetracycline or derivatives thereof or any other inducible promoter. Non-inducible promoters may be CMV—human mammalian cytomegalovirus strong promoter, EF1—human mammalian elongation factor 1 alpha strong promoter, SV40—mammalian simian virus 40 promoter, PGK1—mammalian phosphoglycerate kinase gene promoter, UbC—human mammalian ubiquitin C gene promoter, human beta actin—mammal beta actin gene promoter, CAG—mammalian hybrid promoter or any other non-inducible promoter.

In a preferred embodiment, a DNA construct is provided comprising: the nucleotide sequence encoding a P1 polyprotein; a coumate-inducible promoter operably linked to the nucleotide sequence encoding 3C protease, wherein the nucleotide sequence encoding 3C protease comprises a translation start codon ACG sequence.

Vectors and cells transformed with the DNA construct are provided for stably producing empty capsids of the foot-and-mouth disease virus comprising: a) a nucleotide sequence encoding a P1 polyprotein, b) a nucleotide sequence encoding a 3C protease and c) at least one inducible promoter, wherein the nucleotide sequence encoding the 3C protease comprises a modified translation start codon. In a preferred embodiment, the cell is a mammalian cell, for example, CHO cells that grow in suspension. The inducible promoter may be CR5—coumate-inducible promoter, CR1—coumate-inducible promoter and TRE—tetracycline-inducible promoter or derivatives thereof or any other inducible promoter.

A process for producing empty capsids of the foot-and-mouth disease virus is provided, comprising the following steps:
a. transfecting cells with the above-mentioned construct;
b. selecting the specific clones
c. growing the cells in the presence or absence of an expression inducer; and
d. recovering the empty capsids.

In one embodiment, the cells of step a may be CHO, CHO^{BRI}, CHO-K1, BHK, COS-7, Vero, MDBK, MDCK, NSO cells, or others.

The invention also provides the use of the above-mentioned construct for preparing a vaccine composition against the foot-and-mouth disease and the so prepared composition, wherein said composition may comprise excipients and/or adjuvants.

### BRIEF DESCRIPTION OF THE DRAWINGS

FIG. 1 shows a schematic representation of the VFA constructs included in the plasmids used in the examples;

FIG. 2A shows results of cell viability during the cell selection process with 50 μM methionine sulfoximine (MSX) starting from transfection day (day 0);

FIG. 2B shows results of cell viability during the cell selection process with 25 μM methionine sulfoximine (MSX) starting from transfection day (day 0);

FIG. 3 shows Western Blot results of the first induction assay performed with stable cell cultures selected in the first assay. Cell lysate analysis;

FIG. 4 shows Western Blot results of the second induction assay performed with stable cell cultures selected in the first assay. Cell lysate analysis;

FIG. 5A shows results of cell viability during the cell selection process No. 2 without 10% DymR with 50 μM MSX starting from transfection day (day 0) in the second attempt to obtain stable cell cultures, where constructs with disbalanced expression of polyprotein P1 and protease 3C were used;

FIG. 5B shows results of cell viability during the cell selection process No. 2 with 10% DymR with 50 μM MSX starting from transfection day (day 0) in the second attempt to obtain stable cell cultures, where constructs with disbalanced expression of polyprotein P1 and protease 3C were used;

FIG. 6 shows Western Blot results of the first induction assay performed with stable cell cultures selected in the second assay, which was performed to obtain stable cell cultures, where constructs with disbalanced expression of polyprotein P1 and protease 3C were used;

FIG. 7 shows Western Blot results of the lysates obtained with the stable cell cultures obtained with plasmid pTT81-P1-CR1-ACG-3C at harvest times 0-, 1-, 2-, and 3-days post-induction (dpi) in cultures induced with 2 μg/ml coumate;

FIG. 8 shows results from an ELISA assay on lysates obtained with the stable cell culture obtained with plasmid pTT81-P1-CR1-ACG-3C with the addition of pTT81-CMV-CymR at harvest times 0-, 1-, 2-, and 3-days post-induction (dpi) in cultures induced with 2 μg/ml coumate;

FIG. 9 shows results of a Western Blot assay of lysates from stable cell cultures obtained with the plasmid pTT81-P1-CR1-ACG-3C with and without the addition of pTT81-CMV-CymR, induced by two concentrations (cc) of coumate (2 y 5 μg/ml) and at harvest times 0-, 1-y 2-days post-induction (dpi);

FIG. 10A shows results of a 15-45% sucrose gradient. The pTT81-P1-CR1-ACG-3C-2 and pTT81-P1 -CR1-ACG-3C-5 curves correspond to lysates from stable cell cultures obtained with the construct pTT81-P1-CR1-ACG-3C after 2 days of induction with 2 μg/ml and 5 μg/ml coumate;

FIG. 10B shows results of a 15-45% sucrose gradient. The pTT81-P1-CR1-ACG-3C+10% pTT81-CMV-CymR-2 and pTT81-P1-CR1-ACG-3C+10% pTT81-CMV-Cym R-5 curves correspond to lysates from stable cell cultures obtained with construct pTT81-P1-CR1-ACG-3C and the addition of 10% pTT81-CMV-CymR after 2 days of induction with 2 μg/ml and 5 μg/ml coumate;

FIG. 11 shows the Absorbance obtained from serum analysis at 1/160 dilution for mice vaccinated with 0.5 μg VLPs obtained recombinantly in inducible, transiently transfected CHO mammalian cells, and control mice vaccinated with physiological solution. Sera were analyzed by an ELISA that allows the measurement of specific mouse serology against VFA A2001;

FIG. 12 shows a comparison of the current production of the inactive vaccine, the stable expression achieved by the present construct, and the transient expression previously reported by the inventors; and

FIG. 13 shows a Western Blot assay of cell lysates from CHO cells transiently transfected, and in independent assays, with polyprotein P1 encoding plasmids from various strains and chimeras of VFA (Chimera AA 1-3, A-A2001 4-8, A-Asia1 9-11 and chimera AO 12-14) and protease 3C of serotype A A2001, where cleavage of P1 in VP0 and VP1 corresponding to VFA serotype A2001, VFA serotype A-Asia1, Chimera viral AA and chimera viral AO by protease 3C of A2001, can be observed.

### DETAILED DESCRIPTION OF THE INVENTION

The different expression technologies have limitations when it comes to consider the production of a vaccine against the foot-and-mouth disease, in particular due to the great amount of doses required to meet market needs.

An inducible promoter, for example, coumate together with the use of a 3C protease with a mutation of its start codon of ATGxACG allows for developing stable lines which express higher levels and yields of empty capsids as compared to other heterologous expression systems.

### Definitions

P1 may have the nucleotide sequence of SEQ ID NO:1 corresponding to the modified wild type sequence comprising expression codons in mammalian cells or any P1 sequence of any serotype of the foot-and-mouth disease virus, or modified or optimized sequences to be expressed in mammalian cells. In one embodiment, the nucleotide sequence encoding a P1 polyprotein has a sequence identity to SEQ ID NO:12 from 70% to 99%, or from 90 to 99% to SEQ ID NO:1. More specifically, the nucleotide sequence encoding a P1 polyprotein has a sequence identity of at least 70%, at least 75%, at least 80%, at least 85%, at least 90%, at least 95%, at least 96%, at least 97%, at least 98%, or at least 99% to the full-length sequence of SEQ ID NO:12.
3C may have the nucleotide sequence of SEQ ID NO:2, or any known sequence of different serotypes of the foot-and-mouth disease virus, wherein the nucleotide sequence encoding the 3C protease has a sequence identity to SEQ ID NO:2 from 90% to 99%. More specifically, the sequence encoding a 3C protease has a sequence identity of at least 90%, at least 95%, at least 96%, at least 97%, at least 98%, or at least 99% to full-lenght SEQ ID NO:2
Apaf is a weak sequence (internal ribosome entry site (IRES) shown in SEQ ID NO:3
CR1 and CR5 are coumate-inducible promoters and are shown in SEQ ID NO:4 and SEQ ID NO:5, respectively.
CMV is a cytomegalovirus promoter
CymR is a transcriptional regulator (Poulain et al., 2017, Journal of Biotechnology 255; 16-27)

VLPs or empty capsids are a virus-like particle. These particles are similar to a virus of, in this case, the foot-and-mouth disease but they are not infectious because they lack the viral genetic material.

As used herein, the term “modified start codon” refers to a translation start codon that is different from AUG (or, when referring to a DNA sequence, ATG). Examples of modified start codons that may start the translation in eukaryotes are provided in Kearse et al, Genes & Development, 2017, 31: 1717-1731, and include CUG, GUG, UUG, ACG, AUC, AUU, AAG, AUA, and AGG. The efficiency of translation start with a modified codon typically is lower than translation start efficiency when the start codon is AUG. From Kearse et al. (supra), it would appear that CUG generally is the most efficient start codon; followed by GUG, ACG, and AUU.

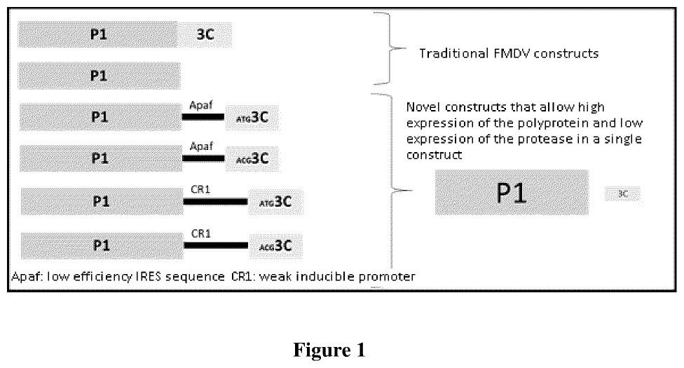

The following plasmids were constructed. They are summarized in FIG. 1
1. pTT81-P13C
2. pTT81-P1
3. pTT81-P1 Apaf ATG 3C
4. pTT81-P1 Apaf ACG 3C
5. pTT81-P1-CR1 ATG 3C
6. pTT81-P1-CR1 ACG 3C
7. pTT81-CMV-CymR

All plasmids were sequenced.

ATG 3C refers to the natural 3C sequence of SEQ ID NO:2 and ACG 3C refers to the mutated 3C sequence, where the translation start codon ATG was changed to the ACG codon.

Two experiments were carried out with the constructed plasmids to develop stable cell cultures.

The first experiment was performed with the plasmids pTT81-P13C and pTT81-P1. Six attempts to obtain stable cell cultures were carried out using these plasmids. On one hand, two concentrations of the selection agent were used, 25 and 50 μM of MSX.
i. On the other hand, the following combinations of plasmids were used: pTT81-P13C, pTT81-P1 and 95% pTT81-P1+5% pTT81-P13C (a combination that was selected because in transient expression experiments that combination resulted in increased yields of empty capsids in CHO cells).

That is, the following attempts were made:

Stable cell cultures obtained using a concentration of 50 μM of MSX:
1. 100% pTT81-P13C
2. 100% pTT81-P1
3. 95% pTT81-P1+5% pTT81-P13C

Stable cell cultures obtained using a concentration of 25 μM of MSX:
1. 100% pTT81-P13C
2. 100% pTT81-P1
3. 95% pTT81-P1+5% pTT81-P13C

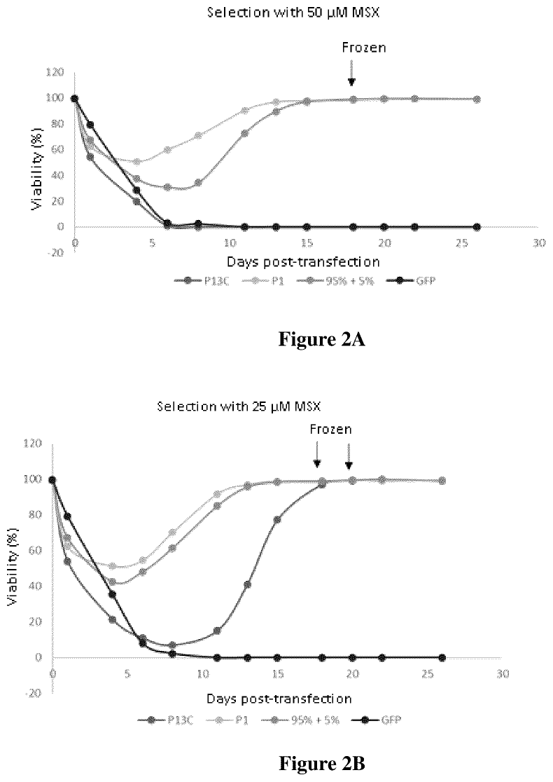

In both cases, a parallel process was also performed using the plasmid pTT0-GFP that lacks the selection gene present in pTT81 and functions as a process control. In Table 1 and FIG. 2 cell viability from the day of transfection (day 0) and during the selection process with MSX is shown.

TABLE 1
Cell viability in the cell selection process with
MSX from the day of transfection (day 0).

| | Viability (%) | | | | | Viability (%) | | | |
|---|---|---|---|---|---|---|---|---|---|
| | 50 um MSX | | | | | 25 um MSX | | | |
| | | | 95% + | | | | | 95% + | |
| | P13C | P1 | 5% | GFP | | P13C | P1 | 5% | GFP |
| 0 | 99.5 | 99.5 | 99.5 | 99.5 | 0 | 99.5 | 99.5 | 99.5 | 99.5 |
| 1 | 54.1 | 62.3 | 67.2 | 79.2 | 1 | 54.1 | 62.3 | 67.2 | 79.2 |
| 4 | 19.8 | 50.8 | 37.7 | 79.2 | 4 | 19.8 | 50.8 | 37.7 | 79.2 |
| 6 | 1 | 60 | 30.9 | 3 | 6 | 11 | 54.6 | 48.1 | 8 |
| 8 | 0 | 70.7 | 34.2 | 2.3 | 8 | 7 | 70.1 | 61.4 | 2.3 |
| 11 | 0 | 90.4 | 72.6 | 0 | 11 | 15 | 91.7 | 85.1 | 0 |
| 13 | 0 | 96.8 | 89.4 | 0 | 13 | 41 | 96.9 | 95.6 | 0 |
| 15 | 0 | 97.6 | 96.8 | 0 | 15 | 77.1 | 98.7 | 98.4 | 0 |
| 18 | 0 | 98.5 | 98.6 | 0 | 18 | 97 | 99.1 | 98.7 | 0 |
| 20 | 0 | 98.9 | 99.3 | 0 | 20 | 99.2 | 99.5 | 99.5 | 0 |
| 22 | 0 | 99.2 | 99.3 | 0 | 22 | 99.7 | 98.9 | 99.7 | 0 |
| 26 | 0 | 99 | 99 | 0 | 26 | 99.1 | 99.5 | 99.2 | 0 |

Once the selection process concluded, two processes were carried out: on one hand, stable cell cultures were frozen for further cell cloning if promisory results were obtained and, on the other, induction assays were carried out with the stable cell cultures and protein expression was analyzed by Western Blot.

It should be noted that with plasmid pTT81-P13C and 50 μM of MSX no clone could be selected. That is, all cells died during the selection process. In the case of plasmid pTT81-P13C and 25 μM of MSX, cultures could be recovered although extremely low viabilities were achieved.

These results are attributable to the expression of the 3C protease, since although the system is inducible there is always a base expression that may affect the selection process in case the protein is toxic and does not lead to stable cultures.

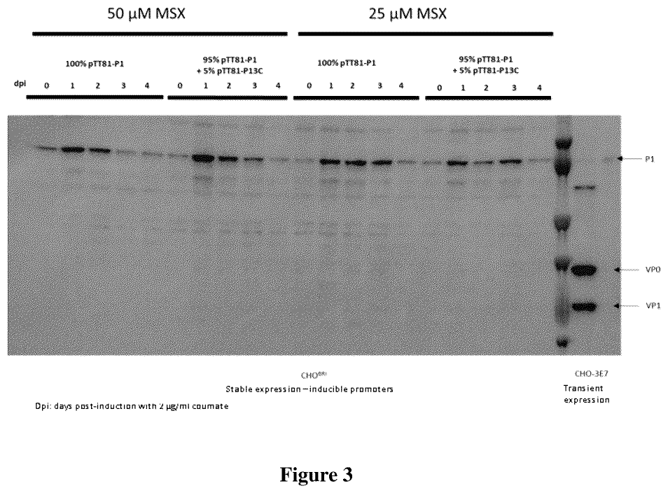

The first induction assay was carried out with the stable cell cultures obtained after transfection with pTT81-P1 and with the combination of 95% pTT81-P1 and 5% pTT81-P13C, both selected with 25 as well as 50 μM of MSX. Cells were seeded at 0.5×10⁶ cells/mL 15 days post-transfection (dpt) and were induced 18 dpt with 2 μg/mL of coumate. Samples were taken the day of the induction and 1, 2, 3, and 4 days post-induction (dpi). Samples were processed to extract proteins from the cells. In addition, cell lysates were analyzed by Western Blot (FIG. 3).

As shown in FIG. 3, none of the constructs were capable of cleaving the polyprotein which is the critical requirement for empty capsid formation. In case of the pTT81-P1 this was foreseeable as the 3C protease was not used in the assay, but in the case of a plasmid mixture of 95% pTT81-P1 and 5% pTT81-P13C it was expected to obtain VLPs. The results suggest that only clones where only plasmid pTT81-P1 was correctly integrated into expression zones were selected.

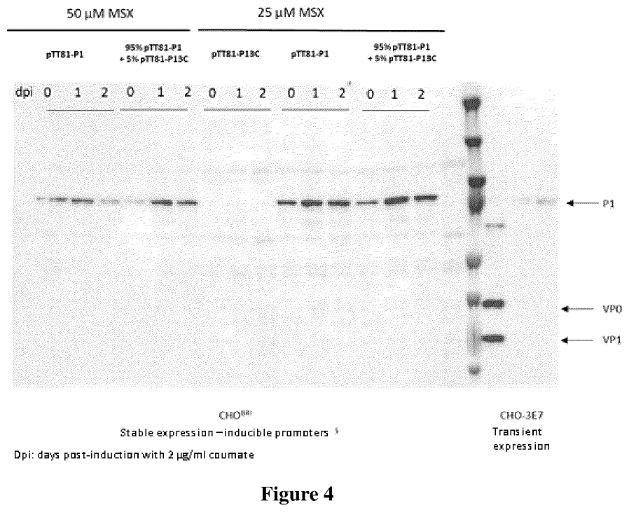

The second induction assay was carried out with all the stable cell cultures obtained after transfection with pTT81-P1 and with the combination of 95% pTT81-P1 and 5% pTT81-P13C, selected with 25 as well as 50 μM of MSX and with pTT81-P13C only selected with 25 μM of MSX. Cells were seeded at 1×10⁶ cells/mL 18 dpt and induced 20 dpt with 2 μg/mL of coumate. Samples were collected on the day of induction and 1 and 2 dpi. Samples were processed to extract proteins from the cells. In addition, cell lysates were analyzed by Western Blot (FIG. 4).

Again, it was only possible to detect a P1 polyprotein by Western Blot both in cells transfected with pTT81-P1 as well as in cells transfected with the combination of 95% pTT81-P1 and 5% pTT81-P13C.

In the case of a stable culture selected by transfection with plasmid pTT81-P13C it was not possible to detect specific bands by Western Blot. However, after analyzing the samples by ELISA it was possible to detect the specific protein 1 dpi but with a very low yield (100 ng/mL).

Interestingly, it is worth noting that a base expression of about 10 ng/mL could be detected by ELISA on the day of induction in stable cultures obtained with the plasmid pTT81-P13C. This might indicate that the cells only tolerate a very low base expression of 3C protease. In view of these results, subsequent approaches of stable cell cultures were performed in such a way that expression of 3C protease was reduced and toxicity levels tolerated by the cells were achieved.

In order to obtain FMDV VLPs, it was essential to work with a construct comprising in the same plasmid both the polyprotein and the 3C protease but regulated in such a manner that more P1 polyprotein than 3C protease was obtained, so that the level of 3C protease would be sufficiently low as to be tolerated by the cells during the selection process and in the induction assays, but at the same time high enough to achieve a complete cleavage of the P1 polyprotein. In these cases, plasmids pTT81-P13C, pTT81-P1 Apaf ATG 3C, pTT81-P1 Apaf ACG 3C, pTT81-P1-CR1 ATG 3C, pTT81-P1-CR1 ACG 3C were used. These plasmids were designed to express P1 polyprotein and 3C protease in unequal amounts. In turn, since it was necessary to decrease base expression levels of 3C protease, plasmid pTT81-CMV-CymR was also constructed encoding a repressor under a constitutive promoter, but in a plasmid with MSX as selection agent. Increasing the repressor levels could help to decrease the base expression of the system and achieve production of stable cell cultures expressing FMDV empty capsids. This repressor was already expressed in the CHO^{BRI} cell line, but this strategy allows for increasing its levels. In this assay, the process was performed using a single concentration of 50 μM of MSX and, as in the previous experiment, a parallel process was carried out with the pTTO-GFP plasmid that does not have the selection gene present in pTT81 and that acts as a process control.

In brief, the following experiments were carried out, with transfection of CHO cells using the following plasmids and plasmid combinations:
1. pTT81-P13C
2. pTT81-P1 Apaf ATG 3C
3. pTT81-P1 Apaf ACG 3C
4. pTT81-P1-CR1 ATG 3C
5. pTT81-P1-CR1 ACG 3C
6. pTT81-P13C+10% pTT81-CMV-CymR
7. pTT81-P1 Apaf ATG 3C+10% pTT81-CMV-CymR
8. pTT81-P1 Apaf ACG 3C+10% pTT81-CMV-CymR
9. pTT81-P1-CR1 ATG 3C+10% pTT81-CMV-CymR
10. pTT81-P1-CR1 ACG 3C+10% pTT81-CMV-CymR
11. pTT0-GFP

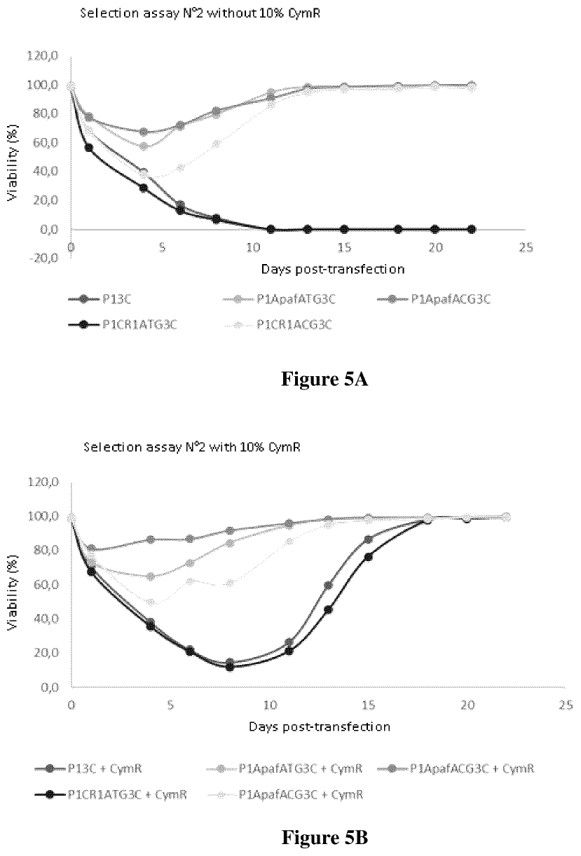

Cell viability of the cultures was tested during the selection process; see Table 2 and FIG. 5.

TABLE 2
Cell viability in the cell selection process with
MSX from the day of transfection (day 0).

| | Viability (%) | | | | | | | | | | |
|---|---|---|---|---|---|---|---|---|---|---|---|
| | 50 um MSX | | | | | | | | | | |
| | 1 | 2 | 3 | 4 | 5 | 6 | 7 | 8 | 9 | 10 | 11 |
| 0 | 98.8 | 98.8 | 98.8 | 98.8 | 98.8 | 98.8 | 98.8 | 98.8 | 98.8 | 98.8 | 98.8 |
| 1 | 68.3 | 78.4 | 77.4 | 56.7 | 68.3 | 70.7 | 73.1 | 81.1 | 67.6 | 77.4 | 75.0 |
| 4 | 39.4 | 57.5 | 67.6 | 28.7 | 37.8 | 38.2 | 64.9 | 86.2 | 35.6 | 50.0 | 54.1 |
| 6 | 17.0 | 71.2 | 71.9 | 13.0 | 43.3 | 21.9 | 72.7 | 86.6 | 20.8 | 62.1 | 9.7 |
| 8 | 7.8 | 79.5 | 82.0 | 6.6 | 59.4 | 14.6 | 84.4 | 91.5 | 11.9 | 61.2 | 2.8 |
| 11 | 0.0 | 94.7 | 90.5 | 0.0 | 86.1 | 26.3 | 94.5 | 95.7 | 21.0 | 85.4 | 0.0 |
| 13 | 0.0 | 98.6 | 97.0 | 0.0 | 94.7 | 59.6 | 98.2 | 98.0 | 45.2 | 95.2 | 0.0 |
| 15 | 0.0 | 98.7 | 98.5 | 0.0 | 96.9 | 86.3 | 98.7 | 99.1 | 76.2 | 97.5 | 0.0 |
| 18 | 0.0 | 99.1 | 99.2 | 0.0 | 97.2 | 97.8 | 99.3 | 98.8 | 97.4 | 98.5 | 0.0 |
| 20 | 0 | 99.6 | 99.3 | 0 | 98.7 | 98.7 | 99.4 | 99.4 | 98.5 | 99.2 | 0 |
| 22 | 0 | 99.7 | 99.7 | 0 | 97.7 | 99.2 | 99.8 | 99.8 | 99.3 | 99.5 | 0 |

After the selection process, two procedures were carried out as in the first selection assay. On one hand, stable cultures were frozen for their conservation and subsequent cell cloning and, on the other, stable cultures were induced and protein expression was analyzed.

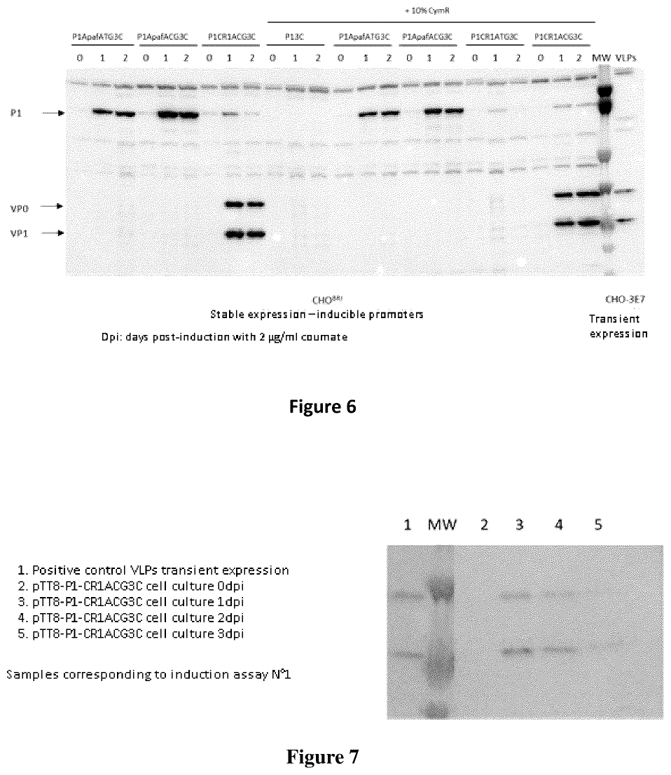

The first induction assay was carried out with the stable cultures that survived the selection process. Cells were induced with 2 μg/mL of coumate. Samples were taken on the day of the induction and 1, 2 and 3 dpi. Samples were processed to extract proteins from the cells and the cell lysates were analyzed by Western Blot. In the first Western Blot only times 0, 1 and 2 dpi were analyzed (FIG. 6).

FIG. 6 shows that only the P1 -CR1 -ACG3C construct (with and without addition of repressor) yielded VP0 and VP1 bands corresponding to a correct processing of P1 by the 3C protease. The mutation of the start codon of 3C protease, by replacement of ATG×ACG, added to the weak inducible promoter CR1, were critical to achieve the protease levels that yielded the stable cell cultures expressing the empty capsids of the foot-and-mouth disease virus under inducible promoters.

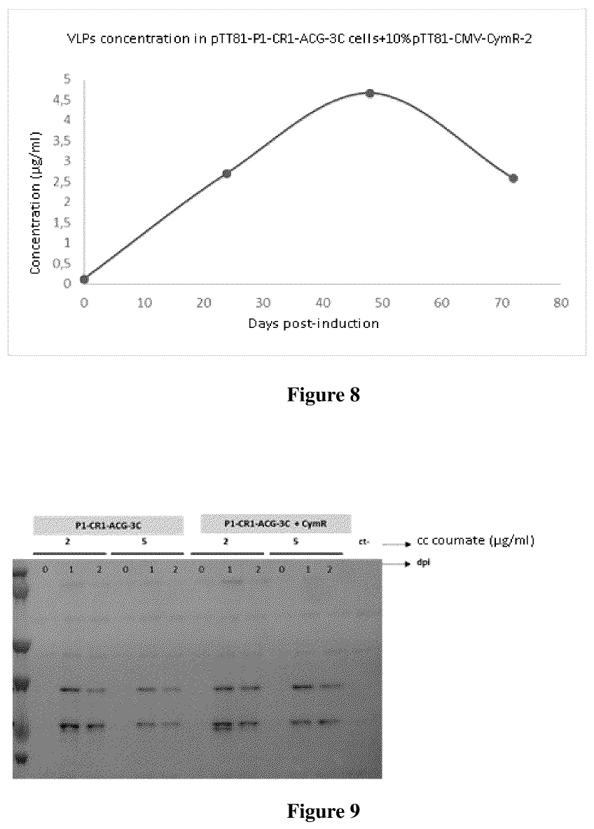

To determine the expression curve of VLPs in the system of stable inducible cell cultures, lysates of culture No. 5 (P1-CR1-ACG3C) were analyzed by Western Blot at harvest times of 0, 1, 2 and 3 dpi (FIG. 7) and lysates of culture No. 10 (P1-CR1-ACG3C+10% CymR) by ELISA, which were obtained from cultures induced with 2 ug/mL of coumate (FIG. 8).

In an additional experiment, the stable cell cultures obtained with the plasmid pTT81-P1-CR1-ACG-3C, with and without the addition of pTT81-CMV-CymR, were induced with two inducer concentrations (2 and 5 μg/mL of coumate) to determine if an increase of inducer concentration had any effect on the achieved expression levels (FIG. 9). This especially applies to the case of adding 10% of plasmid pTT81-CMV-CymR for the transfection since an increase of repressor might require higher levels of inducer to obtain the highest expression levels. However, the addition of a larger amount of inducer had no significant effects on the expression levels of the FMDV VLPs.

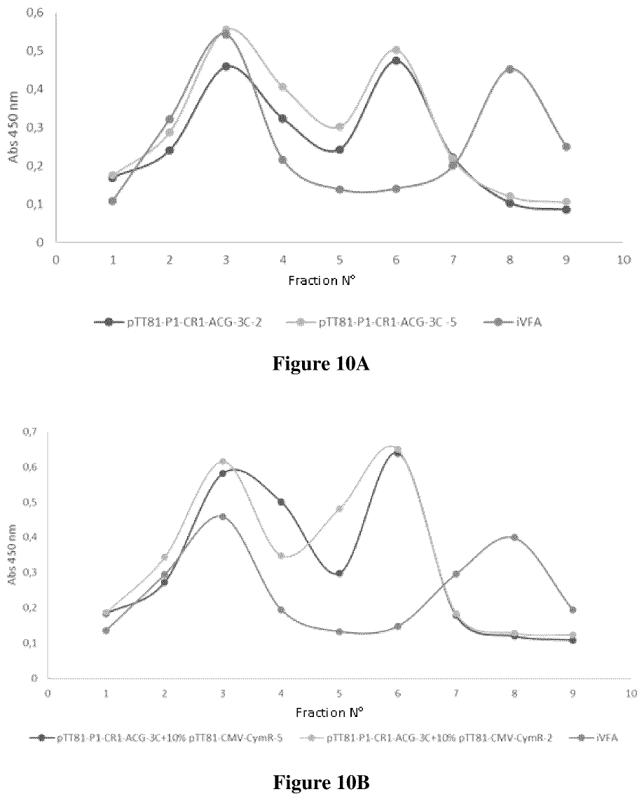

The cell lysates obtained in the induction assays using the stable cell cultures obtained with construct pTT81-P1-CR1-ACG-3C and with construct pTT81-P1-CR1-ACG-3C and addition of 10% pTT81-CMV-CymR were analyzed by sucrose gradient to evaluate a correct formation of the VLPs (FIG. 10). The curves thus obtained confirmed that empty capsid structures are formed with both stable cultures. Induction with 2 μg/mL of coumate not only led to the best concentration of VLPs in both cultures, but also to the highest ratio of VLPs.

The yield obtained by ELISA in induction assay No. 1 was evaluated at 24 hpi with the stable cell culture obtained with construct pTT81-P1-CR1-ACG-3C and with construct pTT81-P1-CR1-ACG-3C and the addition of 10% pTT81-CMV-CymR (No. 5 and No. 10). Yields thus obtained were of 3.6 and 3.3 μg/mL, respectively, and in other assays using productive and cell cloning strategies, an increase of 3-15× was achieved. (about 10 to 50 μg/mL).

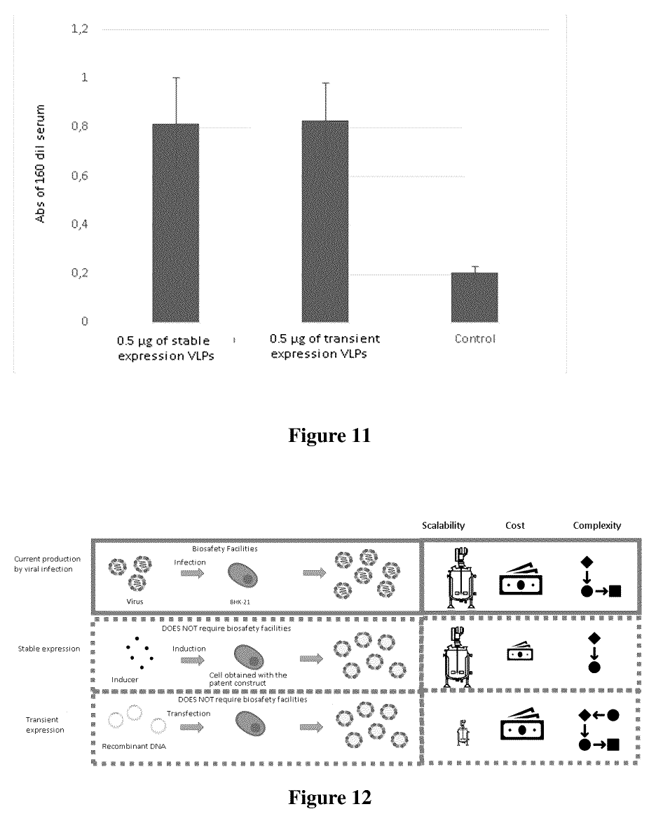

As can be seen in FIG. 11, the use of stably expressed VLPs shows the same activity as that of the ones transiently expressed in an animal model with mice. It may be seen in the figure that the humoral immune response triggered in mice by the recombinant VLPs obtained with the stable system of the invention under inducible promoters is similar to the one triggered by recombinant VLPs obtained by transient expression.

It is important to note that the invention uses mammalian cells as an expression platform. The use of mammalian cells represents an advantage as it implies the use of the platform traditionally employed for producing vaccines against the foot-and-mouth disease, with all the advantages it implies at an industrial level as well as in terms of safety regarding regularly applying the vaccine.

Particularly, the CHO cells that were used grow in suspension in certain fetal bovine serum-free media, where they reach high cell densities, yielding high productivity and low risks associated with the use of products of animal origin. Due to the extensive experience in the biopharmaceutical industry regarding production of recombinant proteins in CHO cells (70% of the approved products are produced in CHO cells), there is great availability and variety of media that may be used to perform the claimed process.

After completing the development, it is only required to grow the cells and add the inducer to start the production. When working with a clonal line, repeatability among batches is granted. FIG. 12 shows a representative stable production scheme of empty capsids using the traditional production of inactivated virus and the transient production previously reported.

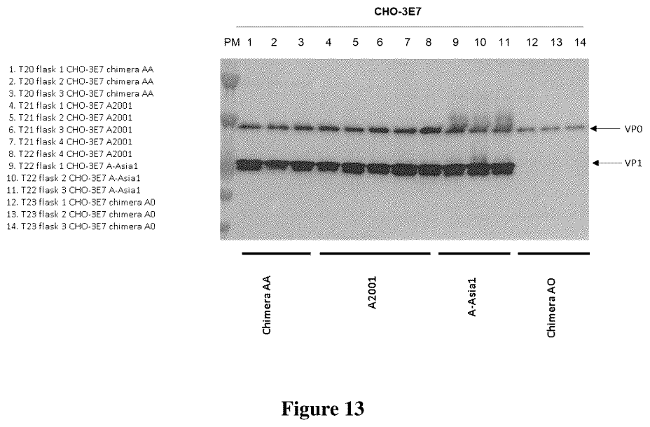

Finally, since the 3C protease is widely conserved among the various FMDV serotypes, the 3C protease gene mutated in the start codon, ATG×ACG, yields stable lines producing empty capsids of all viral serotypes only by replacing the polyprotein in the plasmid used for the transfection. This ability of 3C protease of one serotype to cleave the P1 polyprotein of various serotypes is shown in FIG. 13. FIG. 13 shows how the 3C protease of serotype A A2001 was able to cleave the P1 polyprotein into its structural proteins VP0 and VP1, regardless of the viral strain. It should be noted that the nucleotide sequence encoding the P1 polyprotein may be SEQ ID NO:1, or SEQ ID NO:12 that corresponds to the wild type sequence, or any other sequence encoding a P1 polyprotein.

This invention is better illustrated in the following examples, which should not be construed as a limitation of its scope. On the contrary, it should be clearly understood that after reading the present description other embodiments, modifications and equivalents thereof may be possible, which may be envisioned by a person skilled in the art without departing from the spirit of the present invention and/or the scope of the appended claims.

### EXAMPLES

### Example 1: Cells and Bacteria

In transfection and stable cell selection assays in mammalian cells grown in suspension, CHO^{BRI} cells (cells derived from the CHO DUXB11 cell line adapted to grow in suspension in a serum free medium, and stably expressing the cymene (CymR) repressor and the coumate reverse transactivator (rcTA)), were used. These cells have been created and supplied by NRCC National Research Council of Canada.

DH5α Eschericha coli competent bacteria were used for all cloning procedures and for large scale plasmid production.

Mammalian cell culture: For the culture of cells grown in suspension, the following serum free media were used:

CHO^{BRI}: PowerCHO2 medium (Lonza, Walkersville, MD, USA) and BalanCD Growth A (Irvine Scientific, Irvine, CA USA) supplemented with MSX 25 or 50 μM, CD DG44 medium (Life Technologies Inc., Burlington, ON, Canada), supplemented with 4 mM glutamine and 0.1% Kolliphor® P 188.

Cells were grown in Erlenmeyer flasks of various sizes in an incubator with 5% CO₂ at 37° C. stirred at 120 rpm. For culture maintenance, cell passages were done by dilution.

Count of cells grown in suspension was done by the Trypan blue exclusion method using a CEDEX (Roche, Laval, Qc) cell counter.

D H5α E. coli bacteria were grown at 37° C. in Circle Grown (MP Biomedicals, Solon OH) medium supplemented with 50 μg/ml ampicillin for selecting and growing those clones resistant to that antibiotic. For solid cultures, 2% agar was added to the medium. Petri plates were used for culture in solid medium 90 mm-diameter, and, for liquid culture, 15 mL conic tubes, and 250- and 500-mL Erlenmeyer flaks with constant agitation at 200 rpm, were utilized.

### Example 2: Recombinant DNA Techniques

### Plasmids

To construct those plasmids utilized in the present approach, traditional recombinant DNA techniques, briefly detailed below, were used. Previously synthesized plasmids were used: pTT5-P13C (A2001 wild type sequence) and pTT5-3C (A2001 wild type sequence), synthetic genes from GenScript with sequences optimized for expression in mammalian cells, and genes amplified by PCR [Rowlands D J, Sangar D V, Brown F (1975) J Gen Virol 26:227-238 and S. Misaghi, J. Chang, B. Biotechnol. Prog., 30 (2014), pp. 1432-1440].

TABLE 3
Plasmids used in the assays of stable
expression under inducible promoters

| Plasmid: | Viral serotype: | Feature: |
|---|---|---|
| pTT81-P13C | VFA A2001 | P1 optimized for expression in |
| | | mammalian cells |
| pTT81-P1 | VFA A2001 | P1 optimized for expression in |
| | | mammalian cells |
| pTT81- | VFA A2001 | P1 optimized for expression in |
| P1ApafATG3C | | mammalian cells |
| pTT81- | VFA A2001 | P1 optimized for expression in |
| P1ApafACG3C | | mammalian cells and modified 3C |
| pTT81- | VFA A2001 | P1 optimized for expression in |
| CR5P1CR1ATG3C | | mammalian cells |
| pTT81- | VFA A2001 | P1 optimized for expression in |
| CR5P1CR1ACG3C | | mammalian cells and modified 3C |
| pTT81-CMVCymR | — | Constitutive repressor expression |

### Agarose Gel Electrophoresis

Agarose gels were used for separating DNA samples. For the preparation of 1% agarose gel, the appropriate amount of agarose was weighed and dissolved in TAE buffer (Tris-acetate 10 mM, 1 mM EDTA) using a microwave oven until the agarose was completely melted. Once the solution reached the suitable temperature, a specific dye was added for DNA visualization. This solution was placed on a suitable support with a comb that allowed the generation of lanes for loading the samples. Samples were prepared with 10× loading buffer (50% glycerol, 10× TAE, 1% bromophenol blue). 100 pb and 1 kb molecular weight markers were used.

Electrophoretic runs were performed at room temperature using TAE buffer and applying a voltage of about 100 V. Gels were visualized and registered with Molecular Imager Gel Doc (Bio-Rad) system with the Image Lab (Bio-Rad) software.

### DNA Purification From Agarose Gels

For purifying DNA bands from agarose gels, at the end of the runs, DNA bands of specific sizes were excised by using a scalpel and purified using the QIAEX II Agarose Gel Extraction (QIAGEN) kit, following the manufacturer's instructions. Purified DNA was recovered in a TE buffer solution.

### Plasmid DNA Preparation

In order to obtain high quality plasmids to be used for the transfections, low- and medium-scale DNA preparations were done with commercial Plasmid Purification Mini/midi Kit (Qiagen), following the manufacturer's instructions. Purified DNA was resuspended in sterile bi-distilled water at 65° C.

### Quantification of DNA Fragments and Plasmids

To quantify DNA and to determine the purity of the preparations, a NanoDrop nd-1000 (Thermo Scientific) spectrophotometer was used. For those plasmids to be used in transfections, plasmid purity was assessed by 260/280 nm absorbance (Abs 260/Abs 280) ratio, accepting only those preparations with values in the range of 1.8-2.

### Enzymatic Digestions

DNA digestions with restriction enzymes were performed under the conditions of each enzyme in terms of reaction buffers and temperatures, following the manufacturer's instructions. Approximately 1 to 5 U of enzyme per μg of DNA in a final volume of 20 μl, were used.

### Ligation Reactions

Ligations of DNA fragment to the plasmids were perfomed maintaining a 3:1 ratio of DNA fragment:plasmid in a 10 μl final volume. All reactions were performed with T4 DNA Ligase enzyme and following the manufacturer's instructions.

### Gene Amplification

To construct the following plasmids: pTT81-P1ApafATG3C, pTT81-P1ApafACG3C, pTT81-P1CR1ATG3C, and pTT81-P1CR1ACG3C, PCR reactions were perfomed in order to amplify the fragments: ApafATG3C, ApafACG3C, ATG3C and ACG3C using the primers listed in table 4.

TABLE 4
Primers used in PCR reactions for amplifying
fragments: ApafATG3C, ApafACG3C, ATG3C, and ACG3C.

| Primer name | Primer sequence |
|---|---|
| OG521 | AAACAATTGGGCCGGCCACCATGAGTGGTGCCCCAC |
| SEQ ID No 6 | CGACCG |
| OG522 | AAACAATTGGGCCGGCCACCACGAGTGGTGCCCCAC |
| SEQ ID No 7 | CGACCG |
| OG523 | AAAAGATCTACGCGTTCATTACATCACGTGGACTCG |
| SEQ ID No 8 | TTCTTCCACATCTCTG |
| OG524 | AAAGCGGCCGCTCCACTATTCGAGGCCGTTCGTTAA |
| SEQ ID No 9 | TACTTGTTGCGTTCCTAGCCGCTATT AGGCGCAAA |
| | GGCTTGGCTCATGGTTGACAGCTCAGAGAGAGAAAG |
| | ATCTGAGGGAGGCCGGCCGCCACCATGAGTGGTGCC |
| | CCACCGACCGACCTGCAGAAGATGGTC |
| OG525 | AAAGGATCCTCATTACATCACGTGGACTCGTTCTTC |
| SEQ ID No 10 | CACATCTCTG |
| OG526 | AAAGCGGCCGCTCCACTATTCGAGGCCGTTCGTTAA |
| SEQ ID No 11 | TACTTGTTGCGTTCCTAGCCGCTATTAGGCGCAAAG |
| | GCTTGGCTCATGGTTGACAGCTCAGAGAGAGAAAGA |
| | TCTGAGGGAGGCCGGCCGCCACCACGAGTGGTGCCC |
| | CACCGACCGACCTGCAGAAGATGGTC |

Table 5 describes in detail the PCRs performed and table 6 shows the reaction mixture used for the various reactions.

TABLE 5

| Fragment | Primer F | Primer R | Size | Tm ° C. |
|---|---|---|---|---|
| 1- 3C Apaf ACG | OG526 | OG525 | 836 | 72 |
| 2- 3C Apaf ATG | OG524 | OG525 | 836 | 72 |
| 3- 3C ACG for CR1 | OG522 | OG523 | 731 | 72 |
| 4- 3C ATG forCR1 | OG521 | OG523 | 731 | 72 |

TABLE 6
Reaction mixture used in the PCR.

| PCR Final Vol 50 μl | Vol (μl) | |
|---|---|---|
| 5X Q5 reaction buffer | 10 | μl |
| 10 mM dNTPs | 1 | μl |
| Primer F | 2 μl (final cc 0.2 μM) | |
| Primer R | 2 μl (final cc 0.2 μM) | |
| Template (1/1000 dilution of maxi DNA) | 1 | μl |
| Q5 High-Fidelity DNA Polymerase (NEB) | 0.5 | μl |
| Q5 enhancer | 10 | μl |
| MilliQ water | 23.5 | μl |

### Competent Bacteria Transformation

Competent DH5α bacteria were kept frozen at −70° C. until further use. They were then thawed and kept in ice for 10 min. 2-5 μl of the product of the various ligation reactions or 1 μl of closed plasmid were added to 100 μl of bacteria, gently mixed and ice-cold incubated for 20 min. Subsequently, a thermal shock was applied at 42° C. for 60 s and left on ice for an additional 2 min. Then, 900 μl of LB media were added and incubated for one hour at 37° C. with gentle shaking. Then, tubes were centrifuged at 2000 rpm for 10 min. After centrifugation was completed, 800 μl of the supernatant were discarded and the remaining 200 μl were used for suspending the bacteria that were then plated on LB agar plates supplemented with ampicillin at a final concentration of 100 μg/ml (since this is the bacterial resistance presented by the plasmids used in the present work). Plates were incubated at 37° C. overnight. The resulting colonies were stabbed and inoculated in 3 ml LB/ampicillin liquid cultures and incubated overnight at 37° C.

### Plasmid Sequencing

The appropriate recombinant plasmid construct was confirmed by automatic sequencing of the cloned genes.

### Example 3: Transfections

### Transfection Reagents

Transfections were performed by chemical methods. LPEI-MAX polyethyleneimine (Polysciences) was used for transfecting cells grown in suspension.

Transfection and selection of stable cells from CHO^{BRI} cells grown in suspension.

Cells were prepared for transfection at an appropriate concentration of cells per ml and were transfected only when they showed a viability of more than 95% and appropriate cell densities on transfection day. Both plasmids and PEI were diluted in culture media. The PEI dilution was added to the plasmid dilution, shaken, and incubated at room temperature for a specified time. Then, DNA:PEI mixture was added to the cells. The selection process was initiated with MSX at 24 hpt. For more details about these protocols, see Poulain et al 2017 Journal of Biotechnology 255; 16-27 and Rowlands D J, Sangar D V, Brown F (1975) J Gen Virol 26:227-238. Once the selection process was completed, cell vials were frozen and cultures were prepared to be induced with coumate and to assess the expression. For protein expression analysis, cells were collected by centrifugation and the cell pellet was suspended in lysis buffer (50 mM HEPES, pH 7.4, 150 mM NaCl, 1% Thesit, 0.5% sodium deoxycholate) supplemented with Complete, EDTA-free (Roche) protease inhibitor. Lysis was carried out on ice for 30 min with successive shaking steps for a few seconds. The lysate was then separated from the cell debris by centrifugation at 12.000 g for 10 min at 4° C.

### Fluorescent Protein Expression Analysis

Cells transfected with plasmids codifying for the fluorescent protein were observed under an Olympus IX71 inverted microscope.

A Cellometer (Nexcelom) was used to analyze the “enhanced green fluorescent protein” (EGFP) expression in the assays.

### Example 4: Protein Expression, Quantification, and Structural Analysis of the VFA Recombinant Proteins in CHO^{BRI} Cells

### Recombinant Protein Quantification by ELISA

ELISA plates (Immunolon II) were sensitized overnight at 4° C. with a 1/3000 dilution of an anti-VFA A/Arg/01 rabbit serum in carbonate-bicarbonate buffer pH 9.6. Then, three washes were done with washing solution (0.1% PBS Tween-20) and plates were blocked with blocking solution (0.1% PBS Tween-20, 1% ovalbumin) for 1 hour at 37° C. Then, serial dilutions of assay samples and control samples in blocking solution were added. Serial dilutions in blocking solution of a solution of VFA A/Arg/01 purified by sucrose gradient and quantified taking into account its extinction coefficient were used as standard curve. Both the samples and standard curve were incubated for 1 h at 37 C. After three washes, plates were incubated for 30 min at 37° C. with a 1/3000 dilution of anti-VFA A/Arg/01 guinea pig serum in diluent solution (0.1% PBS Tween-20, 5% normal equine serum). After three washes, anti-guinea pig IgG goat antibodies conjugated with peroxidase enzyme (KPL) diluted in diluent solution were added. Finally, TMB developing solution was added and incubated at room temperature for 20 min. The reaction was quenched with a 12% sulfuric acid solution. Absorbance was read at 492 nm in a Thermo Scientifics Multiskan FC plate reader.

Western Blot: After the addition of 5× Laemmli buffer (60 mM Tris-Cl pH 6.8, 2% SDS, 10% glycerol, 5% mercaptoethanol, 0.01% bromophenol blue) to the samples, they were boiled for 5 min. These prepared samples were loaded on 12% polyacrylamide denaturing gels. Samples were subjected to electrophoresis under a constant voltage of 100 V in running buffer (25 mM Tris-HCl, pH 8.8, 190 mM glycine, 0.1% SDS). At the end of the electrophoretic run, gels were equilibrated with transfer buffer before transferring the separated samples to nitrocellulose membranes. The molecular weight marker used was Precision plus protein TM dual color standard (Biorad). After the transfer, membranes were blocked with a blocking solution at room temperature for 30 min. For immunochemistry detection, anti-VFA A/Arg/01 guinea pig sera diluted 1/500 in blocking solution were used overnight at room temperature. Subsequently, membranes were washed three times with washing solution (PBS, 0.1% Tween-20) for 10 min and then incubated with peroxidase enzyme-conjugated anti-guinea pig IgG antibody (KPL) in blocking solution for 1 h. Membranes were then washed three times with washing solution for 10 min and developed by using a chemiluminescence kit (Biorad).

### Recombinant Structure Characterization by 45-15% Sucrose Gradients

For setting of gradients, in Beckman “Ultra-Clear tubes ½×2 in (13×51 mm)” ultracentrifuge tubes for SW 55 Ti rotor, four PBS solutions with different sucrose concentrations were loaded: 45, 35, 25, and 15% sucrose (W/V). The most concentrated solution was loaded at the bottom of the tube and the most diluted at the top. The loading volume was 1 ml for each solution. Samples were loaded on the top of the gradient. Tubes were centrifuged in a Beckman Optima-LP X-100 Ultracentrifuge with a SW 55 Ti rotor for 2 h at 45000 rpm at 4° C. Acceleration: 9, deceleration: 9. Once the centrifugation was completed, 0.5 mL aliquots were collected. Aliquots were analyzed to detect VFA specific protein by ELISA. Gradient setting and sample collection were done manually.

### Example 5: Mouse Assay

8-week-old female BALB/c inbred strain mice provided by the Animal Facility of the School of Veterinary Sciences of La Plata, Argentina, were used. All laboratory animal experiments were perfomed according to the protocols approved by INTA's Animal Protection Committee. Vaccines were formulated with an oil adjuvant. A 50:50 ratio of antigen:adjuvant was used. BALB/c mice were divided into three groups of five animals each and administered with an intraperitoneal vaccine comprising 50 μg of VLP. At 19-days post-vaccination mice were exsanguinated and serum stored for further analysis.

Sera were analyzed by an ELISA assay that allowed measurement of anti-VFA A001 specific mouse antibodies. Briefly, ELISA plates (Immunolon II) were sensitized overnight at 4° C. with a 1/3000 dilution of an anti-VFA A/Arg/01 rabbit serum in carbonate-bicarbonate buffer pH 9.6. Then, three washing steps with washing solution (PBS 0.1% Tween-20) were done and plates were blocked with blocking solution (PBS 0.1% Tween-20, ovalbumin 1%) for 1 hour at 37° C. Then, a solution with purified and inactivated VFA A/Arg/01 in blocking solution was added before incubating for 1 hour at 37° C. Following three washing steps, plates were incubated for 30 min at 37° C. with various dilutions of mouse sera under evaluation in diluent solution (PBS 0.1% Tween-20, 5% horse normal serum). Following three washing steps, peroxidase enzyme-conjugated goat anti-mouse IgG antibodies (KPL) diluted in diluent solution, were added. Finally, TMB developing solution was added and incubated at room temperature for 20 min. Reaction was stopped with a 12% sulfuric acid solution. Absorbance was measured at 492 nm by means of a Thermo Scientifics MultiskanFC plate reader. The absorbance value is proportional to the level of specific antibodies present in the analyzed mouse serum.

Having thus specifically described and determined the nature and the best mode for carrying out the present invention, we claim property and exclusive right as follows:

1. A DNA construct for producing empty capsids of the foot-and-mouth disease virus comprising:
a) a nucleotide sequence encoding a P1 polyprotein,
b) a nucleotide sequence encoding a 3C protease; and
c) at least one promoter,
wherein the nucleotide sequence encoding the 3C protease comprises a modified translation start codon.

2. The DNA construct according to claim 1, wherein the nucleotide sequence encoding the P1 polyprotein is a sequence having at least 70% identity to SEQ ID NO:12.

3. The DNA construct according to claim 1, wherein the nucleotide sequence encoding the 3C protease is a sequence having at least 90% identity to SEQ ID NO:2.

4. The DNA construct according to claim 1 wherein the nucleotide sequence encoding the P1 polyprotein is a sequence having at least 90% identity to SEQ ID NO:1.

5. The DNA construct according to claim 1, wherein the promoter regulating the 3C protease is a coumate-inducible promoter (CR1).

6. The DNA construct according to claim 1, wherein the promoter regulating the P1 polyprotein is a coumate-inducible promoter (CR5).

7. The DNA construct according to claim 1, wherein the modification of the translation start codon comprises changing ATG by ACG.

8. The DNA construct according to claim 1, further comprising: a coumate-inducible promoter (CR5) operably linked to the nucleotide sequence encoding a P1 polyprotein; a coumate-inducible promoter (CR1) operably linked to the nucleotide sequence encoding a 3C protease, wherein the nucleotide sequence encoding the 3C protease comprises a translation start codon sequence that is ACG.

9. A vector comprising the DNA construct of claim 1.

10. A cell transformed with the vector of claim 9.

11. The cell according to claim 10, wherein is a mammalian cell.

12. A process for producing empty capsids of the foot-and-mouth disease virus comprising the following steps:
a. transfecting cells with the DNA construct of claim 1;
b selecting the specific clones
c. growing the cells; and
d. recovering the empty capsids.

13. The process according to claim 12, wherein in step c. the cells are grown in the presence of an expression inducer.

14. The use of the empty capsids obtained with the process of claim 12, for preparing a vaccine against the foot-and-mouth disease.

15. A vaccine composition comprising empty capsids of the foot-and-mouth disease virus obtained using the process of claim 12; excipients, and/or adjuvants.

16. The composition according to claim 15, wherein the adjuvant is an oil adjuvant.
